(19) United States
(12) Patent Application Publication
(10) Pub. No.: US 2020/0390617 A1
(43) Pub. Date: Dec. 17, 2020

(54) NONWOVEN MICROFIBER PRINT RECEPTIVE SURFACE

(71) Applicant: Kimberly-Clark Worldwide, Inc., Neenah, WI (US)

(72) Inventors: Devin C. McElrath, Broomfield, CO (US); Isabelle R. Bouchard, Fletcher, NC (US); Tim G. Dollevoet, Kimberly, WI (US); Stephen A. Marrano, Oshkosh, WI (US); Samuel B. Henry, Neenah, WI (US)

(21) Appl. No.: 16/608,934

(22) PCT Filed: Jun. 27, 2019
(86) PCT No.: PCT/US19/39494

§ 371 Date: Oct. 28, 2019

Related U.S. Application Data
(60) Provisional application No. 62/691,827, filed on Jun. 29, 2018.

(51) Int. Cl. A61F 13/514; A61F 13/15; B41M 5/50; B41M 5/00

(52) U.S. Cl. CPC A61F 13/51496; A61F 13/15577; A61F 13/51405; B41M 5/502; A61F 2013/51023; B41M 5/0047; A61F 2013/51441; A61F 2013/51439; B41M 5/0011

(57) ABSTRACT

The present disclosure presents a process of forming an aqueous ink onto an ink receptive laminate of a liquid impermeable absorbent article film by ink-jet printing. The process generally includes the steps of corona treating the laminate, applying meltblown fiber onto the laminate and applying ink droplets that are smaller than the meltblown fiber onto the fiber and laminate. An ink-jet printed laminate is also disclosed.

This application claims priority from U.S. provisional Patent Application Ser. No. 62/691,827 filed on 29 Jun. 2018, the entire contents of which are incorporated herein by reference.

### BACKGROUND OF THE DISCLOSURE

Absorbent articles typically include an outercover constructed from a laminate of a liquid impermeable film and a nonwoven fabric constructed from hydrophobic polymeric fibers. These outercovers are difficult to print on in a fast and economical manner that is amenable to efficient ink adhesion and crockfastness. More particularly, it is difficult to get good ink adhesion to the visible surface of the nonwoven web of synthetic fibers.

The need for good ink adhesion to the outercover is enhanced by durability needs of the outercover. For example, when in use, the outercover of absorbent articles can contact oily compositions (e.g., baby oil, lotions, etc.), which can cause the ink to more easily rub off of the outercover. When the oily composition is on the skin of the wearer, or the skin of a caretaker, the ink may rub off of the outercover and onto the skin. Thus, the ink causes unwanted ink stains on the skin of the wearer and/or the caretaker, as well as deteriorate the overall look of the outercover.

In order to protect a design applied to an outer cover, the designs have been printed onto the underlying film layer of the outercover, so that the overlying nonwoven polymeric fabric may help protect the design from rubbing off. While the nonwoven polymeric fabric may protect the underlying design, it also reduces the vibrancy of the design's coloring that is apparent on the resulting absorbent article. In order to compensate for this loss in vibrancy, an excess amount of ink has sometimes been applied to the film, which increases the materials cost of the absorbent article. Additionally, this excess ink may require longer drying time, possibly leading to slower production speeds. These challenges may be severely exacerbated when the use of an aqueous ink is required since drying times are longer and result in slower production speeds. Accordingly, there is a need to improve vibrancy of the color applied, reduce material costs and improve production speed printing of aqueous inks onto the visible surface of a nonwoven web.

### SUMMARY OF THE DISCLOSURE

The current invention pertains to a process of applying an aqueous ink onto an ink receptive laminate comprising of a liquid impermeable film by ink-jet printing. The process includes the steps of applying meltblown fiber onto the laminate, corona treating the laminate, and applying ink droplets that are smaller than the meltblown fiber onto the fiber and laminate. An ink-jet printed laminate is also disclosed.

In one embodiment, a process of forming an aqueous ink onto an ink receptive laminate is disclosed. Specifically, a hydrophobic film/nonwoven laminate (aSFL) may be used. By using an aSFL laminate a dot grain may be achieved which demonstrates improved color vibrancy and crockfastness. Both types of laminates are used as a liquid impermeable film by ink jet printing. The total amount of ink printed onto a meltblown web was from about 1 gsm to about 12 gsm. The laminate disclosed herein is the outercover of an absorbent article. The process comprises pretreating the laminate with corona treatment so as to make the laminate temporarily hydrophilic. Corona treatment used herein may decay immediate or may last up to five years depending upon activation. By applying about 2 gsm to about 7 gsm meltblown fiber web onto the ink receptive laminate and applying about 10 nanograms to about 40 nanogram ink droplets onto the meltblown fiber web wherein the ink droplets penetrate through pores of the meltblown fiber web an image, number or letter is created by ink-jet printing onto the laminate or substrate. The laminate or substrate was subsequently characterized wherein the laminate had a crockfastness of about 5.

In another embodiment of the present disclosure, an ink-jet printed laminate is disclosed. The ink-jet printed laminate disclosed herein comprises about 20 Watts per minute/meter² to about 40 Watts per minute/meter² of corona treatment to the laminate and attached nonwoven to make both laminate and nonwoven hydrophilic wherein the nonwoven is about 2 gsm to about 7 gsm meltblown fiber web wherein the average fiber diameter is from about 3 microns to about 800 nanometers. About 10 nanograms to about 40 nanogram sized ink droplets were added wherein the ink droplets penetrate through pores of the meltblown fiber web and then ink-jet printing the ink-jet droplets and meltblown fiber onto the laminate characterized in that the laminate has a crockfastness of about 5.

In a further embodiment, according to the preceding embodiments, wherein a printing process for making a printed absorbent article is disclosed. The printing process disclosed herein may be used as a manufacturing process for making a printed absorbent article.

### BRIEF DESCRIPTION OF DRAWINGS

The disclosure may be more completely understood in consideration of the following detailed description of various embodiments in connection with the accompanying drawing, in which:

FIG. 1 illustrates a partially cut away, top plan view of an absorbent article in a stretched and laid flat condition with the surface of the article that contacts the skin of the wearer facing the viewer.

FIG. 2 shows aqueous ink droplets (A) being deposited onto a laminate (C) and a meltblown fiber web (B) wherein the meltblown fiber web is used to capture and retain the ink droplets (A).

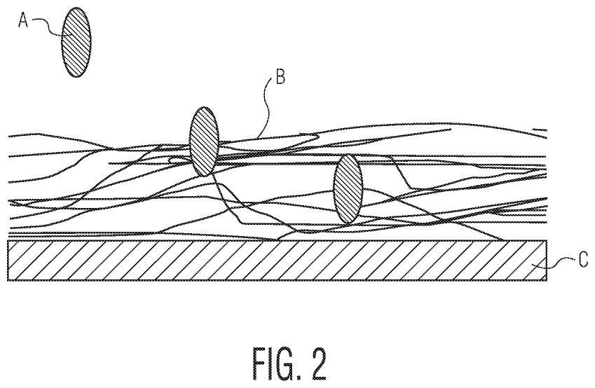

### DETAILED DESCRIPTION OF THE DISCLOSURE

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include diapers, pant diapers, open diapers, diaper covers having fastening means for fastening the diaper, training pants, adult incontinence undergarments, feminine hygiene products, breast pads, care mats, bibs, wound dressing products, and the like. As used herein, the term “body exudates” includes, but is not limited to, urine, blood, vaginal discharges, breast milk, sweat and fecal matter.

The term “absorbent core” for the purposes of the present invention is preferably understood as meaning a construction which in the case of an absorbent article, for instance a diaper, may be arranged between the upper ply, impermeable to aqueous fluids and facing away from the body side of the wearer, and the lower ply, permeable to aqueous fluids and facing the body side of the wearer, and the primary function of which is to absorb and store the fluids, for example blood or urine, which have been imbibed by the absorbent article. The absorbent core itself preferably comprises no imbibition system, no upper ply and no lower ply of the absorbent article.

The term “longitudinal axis” lies in the plane of the article and is generally parallel to a vertical plane that bisects a standing wearer into left and right body halves when the article is worn. The “transverse axis” lies in the plane of the article generally perpendicular to the longitudinal axis.

The term “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwovens may include hydroentangled nonwovens. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

The terms “laminate” or “substrate” include the material that the inks of the present invention may be printed on. Absorbent articles typically include an outercover constructed from a laminate of a liquid impermeable film and a nonwoven fabric constructed from hydrophobic polymeric fibers.

The term “meltblown fibers” refers to fibers that make up a “web” whereby are formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

The term “crockfastness” is a parameter that shows the degree of durability or adhesion of an ink to a laminate or substrate. Crockfastness is measured on a scale from 1 to 5, with 5 being the highest, of the resistance of the material to the transfer of color to another material.

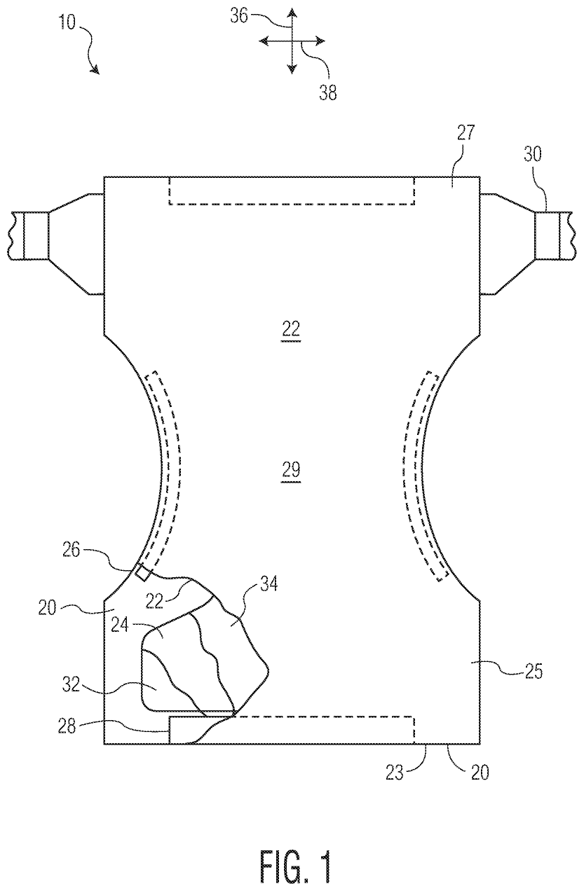

A typical absorbent article will be explained with reference to FIG. 1. FIG. 1 illustrates an exemplary disposable absorbent article 10 that is an infant disposable diaper employing applying an aqueous ink onto an ink receptive laminate or a backsheet (outercover) 20 of an absorbent article 10. The example of the use in a disposable diaper for infants is intended to be representative and not limiting; applying the aqueous ink onto a laminate or outercover of an absorbent article may be used similarly with other types and constructions of absorbent articles. The disposable absorbent article 10 includes a backsheet 20, a liquid permeable topsheet (or bodyside liner) 22 positioned in facing relation with the backsheet 20, and an absorbent core 24, such as an absorbent pad, that is located between the topsheet 22 and the backsheet 20. The article 10 has an outer surface 23, a front waist region 25, a back waist region 27, and a crotch region 29 connecting the front and back waist regions 25, 27. The backsheet 20 defines a length and a width that, in the illustrated aspect, coincide with the length and width of the article 10. The absorbent core 24 generally defines a length and width that are less than the length and width of the backsheet 20, respectively. Thus, marginal portions of the article 10, such as marginal sections of the backsheet 20, can extend past the terminal edges of the absorbent core 24. In the illustrated aspects, for example, the backsheet 20 extends outwardly beyond the terminal marginal edges of the absorbent core 24 to form side margins and end margins of the article 10. The topsheet 22 is generally coextensive with the backsheet 20 but can optionally cover an area that is larger or smaller than the area of the backsheet 20, as desired. In other words, the topsheet 22 is connected in superposed relation to the backsheet 20. The backsheet 20 and topsheet 22 are intended to face the garment and body of the wearer, respectively, while in use.

To provide improved fit and to help reduce leakage of body exudates from the article 10, the article side margins and end margins can be elasticized with suitable elastic members, such as single or multiple strands of elastic. The elastic strands can be composed of natural or synthetic rubber and can optionally be heat shrinkable or heat elasticizable. For example, as representatively illustrated in FIG. 1, the article 10 can include leg elastics 26 that are constructed to operably gather and shirr the side margins of the article 10 to provide elasticized leg bands that can closely fit around the legs of the wearer to reduce leakage and provide improved comfort and appearance. Similarly, waist elastics 28 can be employed to elasticize the end margins of the article 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fastening means, such as hook and loop fasteners 30, may be employed to secure the article 10 on a wearer. Alternatively, other fastening means, such as buttons, pins, snaps, adhesive tape fasteners, cohesives, mushroom-and-loop fasteners, a belt, and so forth, as well as combinations including at least one of the foregoing fasteners can be employed. Additionally, more than two fasteners can be provided, particularly if the article 10 is to be provided in a prefastened configuration.

The article 10 may further include other layers between the absorbent core 24 and the topsheet 22 or backsheet 20. For example, article 10 may also include a surge management layer 34 located between the topsheet 22 and the absorbent core 24 to prevent pooling of the fluid exudates and further improve air exchange and distribution of the fluid exudates within the article 10.

The article 10 may be of various suitable shapes. For example, the article 10 may have an overall rectangular shape, T-shape or an approximately hourglass shape. In the shown aspect, the article 10 has a generally I-shape. The article 10 further defines a longitudinal direction 36 and a transverse direction 38. Other suitable article components that can be incorporated on absorbent articles include containment flaps, waist flaps, elastomeric side panels, and the like. Examples of possible article configurations are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the article 10 are integrally assembled employing various types of attachment mechanisms such as adhesive, sonic bonds, thermal bonds, and so forth, as well as combinations including at least one of foregoing mechanisms. In the shown aspect, for example, the topsheet 22 and backsheet 20 are assembled to the absorbent core 24 with lines of adhesive, such as a hot melt, pressure-sensitive adhesive. Similarly, other article components, such as the elastic members 26 and 28, fastening members 30, and surge layers 34 can be assembled into the article 10 by employing the above-identified attachment mechanisms.

The backsheet 20 of the article 10 may include any material used for such applications, such as a substantially vapor-permeable material. The permeability of the backsheet 20 may be configured to enhance the breathability of the article 10 and to reduce the hydration of the wearer's skin during use without allowing excessive condensation of vapor, such as urine, on the garment facing surface of the backsheet 20 that can undesirably dampen the wearer's clothes. The backsheet 20 can be constructed to be permeable to at least water vapor and can have a water vapor transmission rate of greater than or equal to about 1,000 grams per square meter per 24 hours (g/m²/24 hr). For example, the backsheet 20 can define a water vapor transmission rate of about 1,000 to about 6,000 g/m²/24 hr.

The backsheet 20 is also desirably substantially liquid impermeable. For example, the backsheet 20 may be constructed to provide a hydrohead value of greater than or equal to about 60 centimeters (cm), or, more specifically, greater than or equal to about 80 cm, and even more specifically, greater than or equal to about 100 cm. A suitable technique for determining the resistance of a material to liquid penetration is Federal Test Method Standard (FTMS) 191 Method 5514, dated Dec. 31, 1968.

As stated above, the backsheet 20 may include any material used for such applications, and desirably includes materials that either directly provide the above desired levels of liquid impermeability and air permeability and/or materials that can be modified or treated in some manner to provide such levels. The backsheet 20 can be a nonwoven fibrous web constructed to provide the required level of liquid impermeability. For example, a nonwoven web including spunbond and/or meltblown polymer fibers may be selectively treated with a water repellent coating and/or laminated with a liquid impermeable, vapor permeable polymer film to provide the backsheet 20. In another aspect, the backsheet 20 may include a nonwoven web including a plurality of randomly deposited hydrophobic thermoplastic meltblown fibers that are sufficiently bonded or otherwise connected to one another to provide a substantially vapor permeable and substantially liquid impermeable web. The backsheet 20 may also include a vapor permeable nonwoven layer that has been partially coated or otherwise configured to provide liquid impermeability in selected areas. In yet another example, the backsheet 20 is provided by an extensible material. Further, the backsheet 20 material can have stretch in the longitudinal 36 and/or transverse 38 directions. When the backsheet 20 is made from extensible or stretchable materials, the article 10 provides additional benefits to the wearer including improved fit.

The topsheet 22, employed to help isolate the wearer's skin from liquids held in the absorbent core 24, may define a compliant, soft, non-irritating feel to the wearer's skin. Further, the topsheet 22 can be less hydrophilic than the absorbent core 24, to present a relatively dry surface to the wearer, and can be sufficiently porous to be liquid permeable, permitting liquid to readily penetrate through its thickness. A suitable topsheet 22 may be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials including at least one of the foregoing materials.

Various woven and nonwoven fabrics may be used for the topsheet 22. For example, the topsheet 22 may include a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wettability and hydrophilicity), and the like, as well as combinations including at least one of the foregoing. For example, the topsheet 22 can include a nonwoven, spunbond, polypropylene fabric, optionally including about 2.8 to about 3.2 denier fibers formed into a web having a basis weight of about 22 grams per square meter (g/m²) and a density of about 0.06 gram per cubic centimeter (g/cc).

The absorbent core 24 of the article 10 may include a matrix of hydrophilic fibers, such as a fibrous web of cellulosic fibers, mixed with particles of the particulate superabsorbent polymer composition. The wood pulp fluff can be exchanged with synthetic, polymeric, meltblown fibers, and the like, as well as a combination including at least one of the foregoing. The particulate superabsorbent polymer composition can be substantially homogeneously mixed with the hydrophilic fibers or can be nonuniformly mixed. Alternatively, the absorbent core 24 can include a laminate of fibrous webs and particulate superabsorbent polymer composition and/or a suitable matrix for maintaining the particulate superabsorbent polymer composition in a localized area. When the absorbent core 24 includes a combination of hydrophilic fibers and the particulate superabsorbent polymer, the hydrophilic fibers and particulate superabsorbent polymer composition can form an average basis weight for the absorbent core 24 that may be about 300 grams per square meter (g/m²) to about 900 g/m², or, more specifically, about 500 g/m² to about 800 g/m², and even more specifically, about 550 g/m² to about 750 g/m².

In general, the particulate superabsorbent polymer composition is present in the absorbent core 24 in an amount of greater than or equal to about 50 weight percent (wt percent), or, more desirably greater than or equal to about 70 wt percent, based on a total weight of the absorbent core 24. For example, in a particular aspect, the absorbent core 24 can include a laminate that includes greater than or equal to about 50 wt percent, or, more desirably, greater than or equal to about 70 wt percent of particulate superabsorbent polymer composition overwrapped by a fibrous web or other suitable material for maintaining the high-absorbency material in a localized area.

Optionally, the absorbent core 24 may further include a support (e.g., a substantially hydrophilic tissue or nonwoven wrap sheet (not illustrated)) to help maintain the integrity of the structure of the absorbent core 24. The tissue wrapsheet may be placed about the web/sheet of high-absorbency material and/or fibers, optionally over at least one or both major facing surfaces thereof. The tissue wrapsheet can include an absorbent cellulosic material, such as creped wadding or a high wet-strength tissue. The tissue wrapsheet may optionally be configured to provide a wicking layer that helps to rapidly distribute liquid over the mass of absorbent fibers constituting the absorbent core 24. If this support is employed, the colorant 40 may optionally be disposed in the support, on the side of the absorbent core 24 opposite the backsheet 20.

Due to the thinness of absorbent core 24 and the high absorbency material within the absorbent core 24, the liquid uptake rates of the absorbent core 24, by itself, can be too low, or cannot be adequately sustained over multiple insults of liquid into the absorbent core 24. To improve the overall liquid uptake and air exchange, the article 10 can further include a porous, liquid-permeable layer or surge management layer 34, as representatively illustrated in FIG. 1. The surge management layer 34 is typically less hydrophilic than the absorbent core 24, and can have an operable level of density and basis weight to quickly collect and temporarily hold liquid surges, to transport the liquid from its initial entrance point and to substantially completely release the liquid to other parts of the absorbent core 24. This configuration can help prevent the liquid from pooling and collecting on the portion of the article 10 positioned against the wearer's skin, thereby reducing the feeling of wetness by the wearer. The structure of the surge management layer 34 can also enhance the air exchange within the article 10.

Various woven and nonwoven fabrics may be used to construct the surge management layer 34. For example, the surge management layer 34 can be a layer including a meltblown or spunbond web of synthetic fibers (such as polyolefin fibers); a bonded-carded-web or an airlaid web including, for example, natural and/or synthetic fibers; hydrophobic material that is optionally treated with a surfactant or otherwise processed to impart a desired level of wettability and hydrophilicity; and the like, as well as combinations including at least one of the foregoing. The bonded carded-web can, for example, be a thermally bonded web that is bonded using low melt binder fibers, powder, and/or adhesive. The layer can optionally include a mixture of different fibers. For example, the surge management layer 34 can include a hydrophobic, nonwoven material having a basis weight of about 30 to about 120 g/m².

The backsheet 20 desirably comprises a material that is substantially liquid impermeable, and may be elastic, stretchable or nonstretchable. The backsheet 20 may be a single layer of liquid impermeable material, but desirably comprises a multi-layered laminate structure in which at least one of the layers is liquid impermeable. For instance, the backsheet 20 may include a liquid permeable outer layer and a liquid impermeable inner layer that are suitably joined together by a laminate adhesive (not shown). Suitable laminate adhesives, which may be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. The outer layer may also be made of those materials of which liquid permeable topsheet 22 is made. While it is not a necessity for outer layer to be liquid permeable, it is desired that it provides a relatively cloth-like texture to the wearer.

The inner layer of the backsheet 20 may be both liquid and vapor impermeable, or may be liquid impermeable and vapor permeable. The inner layer is desirably manufactured from a thin plastic film, although other flexible liquid impermeable materials may also be used. The inner layer, or the liquid impermeable backsheet 20 when a single layer, prevents waste material from wetting articles, such as bedsheets and clothing, as well as the wearer and caregiver. A suitable liquid impermeable film for use as a liquid impermeable inner layer, or a single layer liquid impermeable backsheet 20, is a 1.0 mil polyethylene film commercially available from Edison Plastics Company of South Plainfield, N.J., U.S.A. If the backsheet 20 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the backsheet 20. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

The liquid permeable topsheet 22 is illustrated as overlying the backsheet 20 and may but need not have the same dimensions as the backsheet 20. The topsheet 22 is desirably compliant, soft feeling, and non-irritating to the child's skin.

The topsheet 22 may be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics may be used for the topsheet 22. For example, the topsheet may be composed of a meltblown or spunbonded web of polyolefin fibers. The topsheet may also be a bonded-carded web composed of natural and/or synthetic fibers.

The topsheet 22 may be composed of a substantially hydrophobic material, and the hydrophobic material may, optionally, be treated with a surfactant or otherwise processed to impart a desired level of wettability and hydrophilicity. For example, the material may be surface treated with about 0.28 weight percent of a surfactant commercially available from the Rohm and Haas Co. under the trade designation Triton X-102. The surfactant may be applied by any conventional means, such as spraying, printing, brush coating or the like. The surfactant may be applied to the entire topsheet 22 or can be selectively applied to particular sections of the topsheet 22, such as the medial section along the longitudinal centerline.

Alternatively, a suitable liquid permeable topsheet 22 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like. While the backsheet 20 and topsheet 22 may comprise elastomeric materials, it can be desirable in some embodiments for the composite structure to be generally inelastic, where the top sheet, the backsheet 20 and the absorbent core 24 comprise materials that are generally not elastomeric.

In general, the aqueous ink composition as disclosed herein is inhibited from rubbing off of the backsheet (or outercover) such as a film-web laminate 20 and onto the skin of a wearer or a caretaker, even if the outercover 20 contacts an oily substance. Thus, less ink may be applied to the outward facing nonwoven film-web laminate layer of the outercover, when compared to printing on the inner film layer, while still achieving a desired aesthetic design and a desired crockfastness. Furthermore, the color vibrancy of the ink composition printed on the outward facing laminate nonwoven can be retained without being hindered by an overlying layer.

It is increasingly difficulty to utilize aqueous inks with an ink-jet printer in order to achieve increased speed capability. This is because the increased surface area to volume ratio is required to accept a corona treatment and the z-directional, orthogonal to the x and y directions, loft to remove the ink away from a flat surface where the ink may be smeared or disrupted.

A film-web laminate may be formed from a nonwoven web overlying a film layer. In one embodiment, for instance, the nonwoven web is thermally laminated to the film to form the film-web laminate. However, any suitable technique may be utilized to form the laminate. Suitable techniques for bonding a film to a nonwoven web are described in U.S. Pat. No. 5,843,057 to McCormack; U.S. Pat. No. 5,855,999 to McCormack; U.S. Pat. No. 6,002,064 to Kobylivker, et al.; U.S. Pat. No. 6,037,281 to Mathis, et al.; and WO 99/12734, which are incorporated herein in their entirety by reference thereto for all purposes.

The film layer of the laminate is typically formed from a material that is substantially impermeable to liquids. For example, the film layer may be formed from a thin plastic film or other flexible liquid-impermeable material. In one embodiment, the film layer is formed from a polyethylene film having a thickness of from about 0.001 millimeter to about 0.15 millimeters, from about 0.01 to about 0.05 millimeters. For example, a stretch-thinned polypropylene film having a thickness from about 0.001 millimeters to about 0.1. millimeters, or preferably from about 0.01 millimeters to about 0.02 millimeters may be thermally laminated to the nonwoven web.

In addition, the film layer may be formed from a material that is impermeable to liquids, but permeable to gases and water vapor (i.e., “breathable”). This permits vapors to pass through the laminate, but still prevents liquid exudates from passing through the laminate. The use of a breathable laminate is especially advantageous when the laminate is used as an outercover of an absorbent article to permit vapors to escape from the absorbent core, but still prevents liquid exudates from passing through the outer cover. For example, the breathable film may be a microporous or monolithic film.

The film may be formed from a polyolefin polymer, such as linear, low-density polyethylene (LLDPE) or polypropylene. Examples of predominately linear polyolefin polymers include, without limitation, polymers produced from the following monomers: ethylene, propylene, 1-butene, 4-methyl-pentene, 1-hexene, 1-octene and higher olefins as well as copolymers and terpolymers of the foregoing. In addition, copolymers of ethylene and other olefins including butene, 4-methyl-pentene, hexene, heptene, octene, decene, etc., are also examples of predominately linear polyolefin polymers.

### Aqueous Ink Composition

The pigment or dye in the ink composition may be present in an amount effective to provide a visible mark once applied to the laminate. For example, the pigment or dye may be present in the ink composition at concentrations between about 0.25 percent to about 40 percent based on the dry weight basis, and preferably between greater than or equal to about 1 percent and less than or equal to about 10 percent.

Suitable organic pigments, include dairylide yellow MOT (for example, Pigment Yellow 14 CI No. 21095), dairylide yellow AAOA (for example, Pigment Yellow 12 CI No. 21090), Hansa Yellow, CI Pigment Yellow 74, Phthalocyanine Blue (for example, Pigment Blue 15), lithol red (for example, Pigment Red 52:1 CI No. 15860:1). toluidine red (for example. Pigment Red 22 CI No. 12315), dioxazine violet (for example, Pigment Violet 23 CI No. 51319), phthalocyanine green (for example, Pigment Green 7 CI No. 74260), phthalocyanine blue (for example, Pigment Blue 15 CI No. 74160), naphthoic acid red (for example, Pigment Red 48:2 CI No. 15865:2). Inorganic pigments include titanium dioxide (for example, Pigment White 6 CI No. 77891), carbon black (for example, Pigment Black 7 CI No. 77266), iron oxides (for example, red, yellow. and brown), ferric oxide black (for example, Pigment Black 11 CI No. 77499), chromium oxide (for example, green), ferric ammonium ferrocyamide (for example, blue), and the like.

Suitable dyes that may be used with the additive of the present invention include, for instance, acid dyes, and sulfonated dyes including direct dyes. Other suitable dyes include azo dyes (e.g., Solvent Yellow 14, Dispersed Yellow 23, and Metanil Yellow), anthraquinone dyes (e.g., Solvent Red 111, Dispersed Violet 1, Solvent Blue 56, and Solvent Orange 3), xanthene dyes (e.g., Solvent Green 4, Acid Red 52, Basic Red 1, and Solvent Orange 63), azine dyes (e.g., Jet Black), and the like.

The inks are generally dispersed or dissolved in a low viscosity carrier. Exemplary solvents are aliphatic hydrocarbons with common binder types, such as polyamide, shellac, nitro-cellulose, and styrene-maleic. Generally, solvent-based inks include non-catalytic, block urethane resin, which generally demonstrate superior durability over traditional flexographic binders, such as styrene-maleic, rosin-maleic, and acrylic solutions. Desired solvent blends include various acetates such as ethyl acetate, N-propyl acetate, isopropyl acetate, isobutyl acetate, N-butyl acetate, and blends thereof; various alcohols including ethyl alcohol, isopropyl alcohol, normal propyl alcohol, and blends thereof; and glycol ethers including Ektasolve® EP (ethylene glycol monopropyl ether), EB (ethylene glycol monobutyl ether), DM (diethylene glycol monomethyl ether), DP (diethylene glycol monopropyl ether), and PM (propylene glycol monomethyl ether), which may be obtained from Eastman Chemical of Kingsport, Tenn. Other glycols that may also be used are DOWANOL® obtainable from Dow Chemical of Midland, Mich. A desired solvent blend may be a blend of about 50 percent to about 75 percent glycol ether, about 25 percent to about 35 percent N-propyl acetate, and about 15 percent to about 25 percent N-butyl acetate.

Suitable water-based inks that may be used include emulsions that may be stabilized in water-ammonia and may further comprise alcohols, glycols, or glycol ethers as co-solvents. Generally, organic solvents (less than equal to about 7 percent) to water-based inks: alcohols, for example, propan-2-ol may be added for speeding up drying and assisting wetting, glycols, for example, mono propylene glycol to slow down drying, glycol ethers, for example, dipropyl glycol mono methyl ether to aid film formation. Such solvents may be commodity chemicals, commercially available from various companies. Generally, water-based ink includes self-crosslinking acrylic copolymer emulsion, which may have demonstrated superior durability over traditional non-crosslinking binders such as acrylic solutions and dispersion copolymers. Besides the solvent and pigments, the inks may comprise a binder or mixtures thereof. The binder helps stabilize the pigment onto the cover layer 12. Generally, the pigment-to-binder ratios is typically from 1:20 to 1:2.

Waxes may also be included in the ink composition to increase the slip and improve the rub-resistance of the inks of the printed laminate. Common classifications of waxes include animal (for example, beeswax and lanolin), vegetable (for example, carnauba and candellilia), mineral (for example, paraffin and microcrystalline), and synthetic (for example. Polyethylene, polyethylene glycol, and Teflon®). In one embodiment, a wax can be present in an amount of about 0.5 percent to about 5 percent based on the total ink formulation weight when wet.

In one embodiment, the ink compositions used in the printing process to form the indicia are particulate-type ink compositions. The inks chosen should, of course, be safe for human use and should not have environmentally deleterious effects. Moreover, it is desirable that the ink composition is suitable for the intended printing process and is preferably temperature resistant to the process employed for forming the absorbent article, e.g., the temperatures used during a vacuum aperturing process and the like elevated heating processes.

The aqueous ink composition may include a resolubility agent. The resolubility agent may be from about 5.0 to about 16.0 weight percent of the ink composition. Resolubility agents useful in the present invention include acrylics solutions and dispersions with a high to medium degree of carboxyl functionality. In one embodiment, medium acid number, acrylic colloidal dispersion resolubility agents are useful in the present disclosure.

The aqueous ink composition may also include additional waxes and lubricants for detackification. The additional wax/lubricant blend may be comprised of carnauba (the wax) and silicone oil (the lubricant). In one embodiment, the wax/lubricant blend is from about 1 to about 4 weight percent of the composition. Waxes useful in the present invention include polyethylene, polypropylenes, high density polyethylene, low density polyethylene and paraffin.

The aqueous ink composition may include surfactants. Surfactants may be present in the range of from about 1.0 to about 10.0 weight percent of the ink composition. Surfactants useful in the present invention include dioctyl sulfosuccinates, phosphate esters, alkoxylated alcohols, ethoxylated diols, and mixtures or blends thereof.

The aqueous ink composition may be printed on the laminates of a number of article components such as adult feminine care pads, adult, toddler, and infant diapers, including, but not limited to, the backsheet, topsheet, cuffs, etc. of such articles.

Ink-jet printing is a non-impact and non-contact printing method in which an electronic signal controls and directs droplets or a stream of ink that can be deposited on a wide variety of substrates. Ink jet printing is extremely versatile in terms of the variety of substrates that can be treated, as well as the print quality and the speed of operation that can be achieved. In addition, ink jet printing is digitally controllable. For these reasons, ink jet methodology has been widely adopted for industrial marking and labeling.

In another embodiment, the aqueous ink composition may be applied to the laminate by using ink-jet printers. Ink jet ink compositions generally have relatively low viscosities, which enables the ink composition to be applied by spray or throw techniques through the ink jet head onto the substrate. The viscosity of ink jet ink compositions can be from about 0 cP to about 50 cP, such as about 0 cP to about 30 cP, at room temperature (e.g., about 20 degrees centigrade). The ink jet ink compositions can be further subdivided into continuous ink compositions having viscosities of about 0 cP to about 5 cP, and non-continuous ink composition having viscosities of about 0 to about 30 cP.

Ink jet inks are generally from about 2 percent to about 5 percent solids when in the wet state, so applying 2 percent by weight of a crosslinking agent to the wet ink composition results in the dried ink composition containing about 25 percent to about 50 percent by dry weight of the crosslinking agent. Thus, the dried ink composition can contain greater than about 25 percent by weight of the crosslinking agent, such as greater than about 50 percent by weight, when ink jet printed onto the nonwoven web. In some embodiments, the dried ink composition may contain from about 40 percent by weight to about 80 percent by weight, such as from about 50 percent by weight to about 70 percent by weight, when ink jet printed onto the nonwoven web.

Additionally, when applying corona treatment to a laminate and/or nonwoven an increased surface energy that correlates with improved wettability is observed. Corona treatment must be done at an optimal time. If corona treatment is done too long it will destroy the properties of the coating and surface of the substrate. Corona treatment may be performed at various atmospheric pressures. Corona treatment is known in the art of plastic films that generally describes the process of applying an electrical discharge between two narrowly spaced electrodes obtained under atmospheric pressure from a high voltage current. The electrical field generated by the electrodes excites the gas molecules (air) and dissociates some of those molecules to generate a glow of highly energetic species of ions, radicals, metastables and photons. For example, when a polymeric substrate, such as a polyolefin, is passed between two electrodes and is exposed to the glow of active species, changes occur to the polymeric substrate's surface, which usually results in surface oxidation or addition of polar functionalities on the polymeric substrate's surface. These polar functional groups have a strong chemical affinity to the polar chemicals in both the treatment composition as well as in the ink compositions, which results in improved adhesion. Similarly, the more polar polymeric substrate's surface results in an increased surface energy that correlates with improved wettability. For example, the corona treatment may be applied at a level of about 2-50 watts per square foot of web per minute, preferably about 15-40 watts per square foot per minute, more preferably about 8-12 watts per square foot per minute. The aqueous ink receptive laminates of the present invention exhibit better rub resistance, measurable by a higher crockfastness rating (“CR”) compared to what is in the prior art.

### Fiber Diameter Measurements

Fiber diameter measurements were performed via SEM imaging using a high-contrast, back-scatter electron (BE) detector. A BE detector is used when a user scans an ink receptive laminate in back-scatter mode to produce a BE image of the surface. The average fiber diameter range is 3 micron to 800 nanometers. The pore size distribution varies but may be between one micron and 400 nanometers.

### Test Method/Result

Crockfastness test method was used to measure whether the combinations of treated laminates and inks had sufficient abrasion resistance. The crockfastness test method was based upon American Association of Textile Chemists and Colorists (AATCC) Test Method 116-1983, which is incorporated herein in its entirety with a few modifications, as disclosed in international publication no WO 2004 061200A1.

The AATCC Test Method uses a device called a Rotary Vertical Crockmeter to rub a piece of test fabric against the sample specimen. This modified crockfastness test method used a device called at Sutherland Rub Ink Tester (Sutherland 2000 Rubtester supplied by Danilee Company of San Antonio, Tex.) as an alternative to the Crockmeter. The Sutherland Rub Tester is used in the printing industry to evaluate the resistance of inks and coatings on printed substrates. It has a broader test area than the crockmeter. The test head is 2-inchesx4-inches for an eight square inch test area. The test head is moved laterally over the test specimen in a shallow arc pattern. Various weights are available to alter the pressure on the test surface and the number of test “strokes” is variable. This test method used a 4.0 pound weight and 50 rub strokes at a frequency of 42 cycles per minute. The test specimen can be abraded against any material that can be readily attached to the opposing surface of the tester.

Under the AATCC method, any transfer of colorant is qualitatively rated from one to five against a standard scale. A five is equivalent to the absence of transfer and a one is equivalent to an extreme amount of colorant transfer. The primary difference between the test method used in the following examples and the AATCC method was a quantitative method of assigning a colorfastness value. The latter was achieved by using a Spectrodensiometer to assign a measurement of total colorant transfer. This measured value is known as “Delta E”. An equation was then developed to convert the Delta E value to into a one to five value equivalent to the AATCC colorfastness scale.

By corona treating and applying a meltblown fiber web to the laminate and then applying aqueous ink droplets thereon to the meltblown fibers and laminate, the final treated printed laminate may exhibit a superior crockfastness of about 5.0.

### EMBODIMENTS

A first embodiment includes a process of applying an aqueous ink onto an ink receptive laminate of an absorbent article by ink jet printing. The ink receptive laminate comprises pretreating the laminate with corona treatment; applying about 2 gsm to about 7 gsm meltblown fiber web onto the laminate; applying about 10 nanograms to about 40 nanogram sized ink droplets onto the meltblown fiber web wherein the ink droplets penetrate through pores of the meltblown fiber web.

The process according to the preceding embodiment, wherein the laminate is a hydrophobic film/nonwoven laminate (aSFL).

The process according to the preceding embodiments, wherein the absorbent article is packaged for commercial sale.

The process according to the preceding embodiments, wherein the crockfastness is 5.

The process according to the preceding embodiments, wherein the amount of meltblown fiber applied is from about 3 gsm to about 6 gsm.

The process according to the preceding embodiments, wherein the amount of meltblown fiber applied is from about 4 gsm to about 5 gsm.

The process according to the preceding embodiments, wherein the laminate is a liquid impermeable film.

The process according to the preceding embodiments, wherein the printing process disclosed herein may be used as a manufacturing process for making a printed absorbent article.

The process according to the preceding embodiments, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

The process according to the preceding embodiments, wherein the amount of aqueous ink applied to the laminate is dependent on the amount of ink required to prepare a graphic, design or number onto the laminate.

The process according to the preceding embodiments, wherein a design or graphic being printed onto the laminate of the absorbent article is done by sparse printing.

A second embodiment includes an ink-jet printed laminate of an absorbent article comprising about 20 Watts per minute/meter2 to about 40 Watts per minute/meter2 of corona treatment that is applied to the laminate; about 2 gsm to about 7 gsm meltblown fiber web wherein the average fiber diameter is from about 3 microns to about 800 nanometers; about 10 nanograms to about 40 nanogram sized ink droplets and wherein the ink droplets penetrate through pores of the meltblown fiber web.

The article according to the preceding embodiment, wherein the laminate is a hydrophobic film/nonwoven laminate (aSFL).

The article according to the preceding embodiments, wherein the absorbent article is packaged for commercial sale.

The article according to the preceding embodiments, wherein the crockfastness is 5.

The article according to the preceding embodiments, wherein the amount of meltblown fiber applied is from about 3 gsm to about 6 gsm.

The article according to the preceding embodiments, wherein the amount of meltblown fiber applied is from about 4 gsm to about 5 gsm.

The article according to the preceding embodiments, wherein the laminate is a liquid impermeable film.

The article according to the preceding embodiments, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

The article according to the preceding embodiments, wherein the amount of aqueous ink applied to the laminate is dependent on the amount of ink required to prepare a graphic, design or number onto the laminate.

The article according to the preceding embodiments, wherein a design or graphic being printed onto the laminate of the absorbent article is done by sparse printing.

What is claimed is:

1. A process of applying an aqueous ink onto an ink receptive laminate of an absorbent article by ink jet printing comprising:
pretreating the laminate with corona treatment;
applying about 2 gsm to about 7 gsm meltblown fiber web onto the laminate;
applying about 10 nanograms to about 40 nanogram sized ink droplets onto the meltblown fiber web wherein the ink droplets penetrate through pores of the meltblown fiber web.

2. The process of forming an aqueous ink onto a laminate according to claim 1, wherein the laminate is a hydrophobic film/nonwoven laminate (aSFL).

3. The process of forming an aqueous ink onto a laminate according to claim 1, wherein the absorbent article is packaged for commercial sale.

4. The process of forming an aqueous ink onto a laminate according to claim 1, wherein the aqueous ink on the laminate has a crockfastness of 5.

5. The process of forming an aqueous ink onto a laminate according to claim 1, wherein the amount of meltblown fiber applied is from about 3 gsm to about 6 gsm.

6. The process of forming an aqueous ink onto a laminate according to claim 1, wherein the amount of meltblown fiber applied is from about 4 gsm to about 5 gsm.

7. The process of forming an aqueous ink onto a laminate according to claim 1, wherein the laminate is a liquid impermeable film.

8. The process of forming an aqueous ink onto a laminate according to claim 1, wherein the printing process disclosed herein may be used as a manufacturing process for making a printed absorbent article.

9. The process of forming an aqueous ink onto a laminate according to claim 1, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

10. The process of forming an aqueous ink onto a laminate according to claim 1, wherein the amount of aqueous ink applied to the laminate is dependent on the amount of ink required to prepare a graphic, design or number onto the laminate.

11. The process of forming an aqueous ink onto a laminate according to claim 1, wherein a design or graphic being printed onto the laminate of the absorbent article is done by sparse printing.

12. An ink-jet printed laminate of an absorbent article disclosed herein comprising:
about 20 Watts per minute/meter2 to about 40 Watts per minute/meter2 of corona treatment that is applied to the laminate;
about 2 gsm to about 7 gsm meltblown fiber web wherein the average fiber diameter is from about 3 microns to about 800 nanometers;
about 10 nanograms to about 40 nanogram sized ink droplets and wherein the ink droplets penetrate through pores of the meltblown fiber web.

13. The ink-jet printed laminate according to claim 12, wherein the laminate is a hydrophobic film/nonwoven laminate (aSFL).

14. The ink-jet printed laminate according to claim 12, wherein the absorbent article is packaged for commercial sale.

15. The ink-jet printed laminate according to claim 12, wherein the ink-jet laminate has a crockfastness of 5.

16. The ink-jet printed laminate according to claim 12, wherein the amount of meltblown fiber applied is from about 3 gsm to about 6 gsm.

17. The ink-jet printed laminate according to claim 12, wherein the amount of meltblown fiber applied is from about 4 gsm to about 5 gsm.

18. The ink-jet printed laminate according to claim 12, wherein the laminate is a liquid impermeable film.

19. The ink-jet printed laminate according to claim 12, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

20. The ink-jet printed laminate according to claim 12, wherein the amount of aqueous ink applied to the laminate is dependent on the amount of ink required to prepare a graphic, design or number onto the laminate.

21. The ink-jet printed laminate according to claim 12, wherein a design or graphic being printed onto the laminate of the absorbent article is done by sparse printing.
